(19) United States
(12) Patent Application Publication
(10) Pub. No.: US 2005/0053955 A1
(43) Pub. Date: Mar. 10, 2005

(54) Nucleic acid-based assay and kit for the detection of methanogens in biological samples

(76) Inventors: Mohankumar R. Sowlay, Phillipsburg, NJ (US); Stephen M. Hinton, Chester, NJ (US)

Correspondence Address: EXXONMOBIL RESEARCH AND ENGINEERING COMPANY, P.O. BOX 900, 1545 ROUTE 22 EAST, ANNANDALE, NJ, 08801-0900, US

(21) Appl. No.: 10/703,986

(22) Filed: Nov. 7, 2003

Related U.S. Application Data
(63) Continuation-in-part of application No. 09/927,239, filed on Aug. 10, 2001, now abandoned.
Application No. 09/927,239 is a continuation-in-part of application No. 09/838,800, filed on Apr. 20, 2001, now abandoned.

(51) Int. Cl. C12Q 1/68; C07H 21/04

(52) U.S. Cl. 435/6; 536/24.1

(57) ABSTRACT

A nucleic acid based method is provided for the detection of methanogens in human, animal, plant and in environmental samples of soil, sediment or water that are terrestrial or subterranean in origin. The method is effected by (a) obtaining a biological sample; and (b) analyzing the sample for a nucleic acid sequence/s unique to methanogens, wherein a detectable level of the nucleic acid sequence unique to methanogens is indicative of the presence of methanogens in the sample. Further, a scheme for inferring the identity of the different types of methanogens is provided, wherein, the DNA sequences of the methyl reductase genes detected in that sample are compared to methyl reductase sequences of known methanogens. With this technology, methanogens in samples containing less than 1/1000^{th} of a gram of biomass can be detected.

This application is a Continuation-in-Part under 37 CFR 1.53(b) of U.S. application Ser. No. 09/927,239 filed Aug. 10, 2001, which is a Continuation-in-Part of U.S. Ser. No. 09/838,800 filed Apr. 20, 2001.

### BACKGROUND OF THE INVENTION

Methanogens are important members of microbiological consortia in natural environments, subterranean formations including petroleum reservoirs and also in marine and land animals, insects and human gut, peat bogs, waste streams, etc. However, there is no standard method of detecting methanogens. One method of methanogen detection is to culture them (2, 3, 4, 5, 6, 7, 8, 9). Cultivating methanogens anaerobically in a laboratory is a laborious and time-consuming process. Another method of identifying methanogens is to use rRNA targeted archeabacteria specific PCR primers (10, 11) or methanogen specific group-specific 16s rDNA probes (12, 13). These methods suffer from a limitation wherein the probes cross-react with organisms of other physiological, or even phylogenetic groups when applied to environmental samples containing unknown sequences. The present invention is a method for testing the presence of the methanogen specific DNA since the DNA technology has the advantages of speed, accuracy, ease of practice, and low-levels of detection and the method is described below.

A nucleic acid based method is provided for the detection of methanogens in human, animal, plant and in environmental samples of soil, sediment or water that are terrestrial or subterranean in origin. The method is effected by (a) obtaining a biological sample; and (b) analyzing the sample for a nucleic acid sequence/s unique to methanogens, wherein a detectable level of the nucleic acid sequence unique to methanogens is indicative of the presence of methanogens in the sample. Further, a scheme for inferring the identity of the different types of methanogens is provided, wherein, the DNA sequences of the methyl reductase genes detected in that sample are compared to methyl reductase sequences of known methanogens. With this technology, methanogens in samples containing less than 1/1000^{th} of a gram of biomass can be detected.

### SUMMARY OF THE INVENTION

Biotechnologies, including methods to detect nucleic acids, form the foundations of the rapidly evolving and growing biotechnological companies. Nucleic acid based assays and detection methods have widespread application in the detection of specific nucleic acids and thus affects many fields, including human and veterinary medicine, food and agricultural processing and environmental testing.

Alternative methods and products are needed to overcome the limitations imposed by the lack of a technique, cost or availability of reagents or equipment. Furthermore, the ability to introduce a new tool to obtain the accuracy and sensitivity needed for a certain application, to minimize the time spent or the number of steps, to automate a process and to avoid radioactive or other hazardous materials is made possible by innovation of new methods. Specifically, the technical reasons for testing for the presence of the methanogen specific DNA is that the DNA technology has the advantages of speed, accuracy, ease of practice, and low-levels of detection.

There are many applications of the detection of nucleic acids in the art, and a DNA/RNA based detection of methanogen specific nucleic acid is but one of those methods. A major limitation of rRNA-targeted group-specific probes is that they may cross-react with organisms of other physiological, or even phylogenetic groups when applied to environmental samples containing unknown sequences (1). In this invention the restricted physiology of methane-producing bacteria in hydrocarbon bearing subterranean formations is used in identifying them with DNA probes by specifically and efficiently targeting a unique gene specific to the physiology of methanogens that encodes methyl reductase enzyme.

The present invention allows the detection of nucleic acids, including methanogen specific nucleic acids, and is intended to be a portion of, but not limited to, the process of stimulating subterranean microbial activity. The ability to detect and identify microorganisms based on nucleic acid assays is useful especially because culturing many of these microorganisms from natural environments is a time-consuming process ranging from a few days to a few months or even years, or are not culturable at all in the laboratory. The method proposes to detect methyl reductase genes in biological samples obtained from animal, plant, microorganisms, resident or isolated, and part of three states of matter comprising of solids, liquids or air.

In detecting the methanogens, advantage is taken of many technologies, primarily the process of polymerase chain reaction (PCR). The PCR process is well known in the art (U.S. Pat. Nos. 4,683,195, 4,683,202, and 4,800,159). To briefly summarize PCR, nucleic acid primers or oligo-nucleotides, complementary to opposite strands of a nucleic acid amplification target sequence, namely, the methyl reductase gene, are permitted to hybridize to the denatured sample. Typically, a heat stable DNA polymerase extends the DNA duplex from the hybridized primer. Although, the invention is described for the process of PCR, it is not intended to be limited to PCR, but is applicable to many other techniques familiar to one well versed in the art. Thus according to other preferred embodiments the non-exhaustive list of techniques comprise of primer extension, nucleic acid sequence-based amplification (NASBA) and strand displacement amplification (SDA), Cycling Probe Reaction (CPR), Ligase Chain Reaction (LCR) and the related Gapped Ligase Chain Reaction (G-LCR).

In PCR, the repeated cycles of heating and cooling exponentially amplifies the nucleic acid target. In the absence of a (methanogen) target, or the presence of unknown sequences, the oligonucleotide primers will not hybridize resulting in a failure to yield a corresponding amplified PCR product. Thus, the primers behave as hybridization probes.

The specific interaction of the primers with their target sequence leads to correct amplification of the target consistent with the size of internal sequence that corresponds to approximately (144 amino acids) 432 nucleotide basepairs in length in this invention. The specificity of such a reaction is easily ascertained by simple techniques to determine the size of the amplified target sequence. Specifically, PCR products made with unlabeled primers may be detected by electrophoretic gel separation methods followed by dye-based (ethidium bromide, SYBR Green, etc.) visualization. Amplification products are separated to form a ladder in an agarose gel corresponding to a standard marker ladder such as the 50 or 100 base pair ladders which are available commercially. Thus, a desirable difference in length between the reference sequences is 50 or 100 nucleotides and/or multiples there of.

Subsequently, the mixture PCR products called amplicons, from diverse methanogen methyl reductase genes, may be purified, cloned into commercially available molecular vehicles called plasmid DNA, resulting in recombinant plasmid DNA (rDNA). The purpose of cloning is to separate the individual amplicons for subsequent laboratory manipulations including, but not limited to, rDNA purification and DNA sequencing.

The individual amplicons, now in the form of rDNA, can be replicated in competent Escherechia coli, either produced in-house or obtained commercially. Such isolated rDNA is used in DNA sequencing reactions to determine the DNA sequence of the amplified methyl reductase genes. These DNA sequences are then compared to known DNA sequences of methyl reductase genes available in public databases. Such comparison provides the basis for inferring the identity of the unknown methanogens in our sample.

Sensitive and rapid detection of methanogens in environmental samples is important from the viewpoint of exploitation of natural resources, for instance, conversion of unrecoverable petroleum crude oil to methane by stimulating microbial activity in subterranean hydrocarbon formations.

The present invention successfully addresses the shortcomings of the currently known configurations by providing a highly sensitive DNA assay for detecting methanogens in biological samples, especially useful when they are part of microbial consortia, for which no specific assay is so far available.

Supplementation of traditional microscopic examinations with molecular methods provides a cost-effective and rapid technique for detecting specific DNA of methanogens in environmental samples.

Any biological sample is amenable to the nucleic acid-based assay according to the present invention. Of particular importance are environmental samples derived from hydrocarbon bearing formations that are terrestrial or subterranean, anoxic ditch muds, lake or marine sediments, waste streams, etc., prone to methanogen habitation. However, as will be appreciated by one ordinarily skilled in the art, this example is not intended to and should not be considered as limiting.

Specifically, the present invention is used for the detection of methanogens in environmental samples implementing any one of a variety of amplification assays such as PCR or hybridization and/or synthesis molecular techniques that are template dependent. Template is any isolated fragment of nucleic acid, DNA or messenger RNA, that includes a region that has a region of nucleic acid sequence complementary to the nucleic acid probes contemplated in this invention.

According to further features described in the preferred embodiments the template-dependent assay is a template-dependent synthesis assay.

The present invention describes in a preferred embodiment that the template-dependent assay is a template-dependent hybridization assay.

The preferred embodiments of the present invention also include features of the template-dependent assay that is a template-dependent hybridization and synthesis assay.

According to the features described in the preferred embodiments the template-dependent assay includes a step of primer extension effected by at least one oligonucleotide having a sequence hybridizable with the nucleic acid sequence unique to methanogens.

The present invention, of a nucleic acid-based assay, can be used as a kit for the detection of methanogens in any biological sample. Such a kit can include a description of the detection methods of the invention, including detection by fluorescent DNA detectors and the like.

The oligonucleotides above, included in the contemplated kit, are supplied dissolved in water or as lyophilized powder, with or without modifications, at a (certain) concentration (of 1 mg/mL).

As was the case in the previous embodiment, dNTPs in the kit are utilized in the extension reactions. Preferably, these reagents, and all of the reagents utilized in the kits discussed herein, are free of contaminating methanogen DNA or may separately contain a sample of methanogen DNA as a positive control to test the efficacy of the kit components.

In an aspect of the present invention there is provided in the kit, useful for the detection of methanogen presence in a sample. The kit comprises of a carrier being compartmentalized to receive in close confinement therein one or more containers comprising at least one oligonucleotide having a sequence hybridizable with a nucleic acid sequence unique to methanogens.

It is preferred that the polymerase enzyme utilized for an extension reaction be a template-dependent polymerase. According to further features in preferred embodiments of the invention described below, the kit is contemplated to include a template dependent DNA polymerase, preferably a thermostable DNA polymerase. Such polymerases may include Taq polymerase that has the activity or Pfu polymerase that is free of activity of adding a 3′-terminal deoxyadenosine in a template-nonspecific manner.

Where RNA is used as a template in practicing this invention, those reverse transcriptases that are capable of functioning at room temperature or those that are thermostable or those that can be used in RT-PCR applications may also be used.

The extended probe/target hybrid is separated from any unreacted dNTPs, i.e., purified at least to the degree needed to use the extended probe strand to determine the presence or absence of the targeted nucleic acid in the sample or to obtain its sequence.

### BRIEF DESCRIPTION OF THE DRAWINGS

FIG. 1 shows a schematic illustration of reduction of carbon dioxide to methane involving step-wise addition of hydrogen. The terminal step is catalysed by the methyl reductase enzyme.

FIGS. 2A and 2B show an example of amino acid sequence alignment of alpha subunit of methyl reductase enzymes of SEQ ID NOs. 1, 5, 9, 13 and 17. Single letter codes for the amino acids in the methyl reductase protein sequence have been used. The use of single letter codes for the amino acids is well known in the art (32). The conserved positions are marked with an asterisk. In addition, the conserved structure-function domains used for PCR probing are boxed and the direction of PCR extension is marked with an arrow. The solid line represents a non-extendable fluorescently labeled DNA probe. The complete scientific names for the methanogens whose mcrA protein sequences identified as SEQ ID NOs. 1, 5, 9, 13 and 17 are given in FIG. 10.

FIG. 3 shows a line diagram of mcrA gene sequences where the conserved sections with homologous DNA sequence is shown in blocks and the varying sequence of non-conserved sections of mcrA genes from different organisms is shown by lines. Below, a hypothetical section of the gene that is the targeted segment of PCR replication is shown.

FIG. 4 shows the design of the PCR primers MF328 and MR472, represented by SEQ ID Nos 72 and 73 respectively, and the internal probe MC442 represented by SEQ ID No 74. The amino acid sequences represented by the SEQ ID Nos 30 through 45, 46 and 47, and 48 through 71 correspond to different variations of the amino acid domains AA328, AA472 and AA442 respectively. The three letter codes of the amino acids is translated to the three-letter codons to obtain a consensus nucleic acid sequence. Since the target nucleic acid is a duplex molecule of complementary strands, the reverse complement of translated sequence forms the actual reverse PCR primer in the case of MR472. The internal probe MC442 is also designed to anneal to the target 60 base pairs downstream of the reverse primer. The letter code, ‘N’ in the nucleic acid sequence represents any of the four nucleotides, it has been replaced with the nucleotide T because T can base pair with any of the four nucleotides. This substitution of T for N reduces the complexity of the primers and probe. The asterisk represents an art of the protein sequence constituting methyl coenzyme M reductase I alpha subunit of Methanobacterium thermoautotrophicum whose Genbank database accession number is AAA73445 (24) and the sequence is depicted in FIG. 2.

FIG. 5 shows a representative group of methanogens and the range of food substrates for their energy requirement.

FIG. 6 shows an illustration of the natural phenomena of DNA replication that occurs inside every living cell.

FIG. 7 shows the polymerase chain reaction (PCR) is an in-vitro process that amplifies a specified region of DNA by mimicking the natural phenomena of DNA replication. A heat stable DNA polymerase synthesizes a complementary strand of DNA in the 5′ to 3′ direction using one strand as a template in a DNA extension reaction, repetitively. Repeated cycles of heating and cooling allow the added primers to hybridize to the target region in the separated strands of DNA. The DNA polymerase synthesizes new strands of DNA producing many copies of the original segment of the DNA.

FIG. 8 shows a schematic of the cloning process, where DNA fragments are grafted into plasmid DNA and introduced into bacteria. Transformant bacteria with recombinant DNA grow as individual colonies and the bacteria within each colony contain identical rDNA.

FIG. 9 shows a schematic representation of automated DNA sequencing.

FIG. 10 shows a phylogenetic tree of methyl reductase protein-Alpha Subunit. The superscript denotes the SEQ ID NOs.

### DETAILED DESCRIPTION OF THE INVENTION

In petroleum recovery operations, a significant portion, amounting to as much as 75% of the original crude oil reserves remain in subterranean rock formations. Among the enhanced oil recovery techniques targeting the unrecoverable hydrocarbon, the use of microorganisms has found applications primarily in altering the permeability of subterranean formation, producing biosurfactants that decrease surface and interfacial tension and generating gases such as carbon dioxide to increase formation pressure. However, the extreme conditions of high temperature, salinity and anaerobic conditions, to name a few, prevalent in the formation limit the types and numbers of microorganisms and the viability of those exogenously introduced into the formation. Therefore, it has been proposed that indigenous microorganisms be employed to circumvent the problems in microbial enhanced oil recovery processes. In one such recovery process, it has been suggested to use microorganisms in converting fossil fuel deposits to methane since these reservoirs are also home to large amounts of biogenic methane. U.S. Pat. No. 3,826,308 demonstrates this concept by microbial production of methane from fossil fuel deposits containing organic ring compounds. Another U.S. Pat. No. 5,424,195 discloses a method to convert coal to methane in a coal-bearing cavity using exogenous microorganisms. In spite of these and other prior inventions, there is not only no information on how to identify specific microorganisms to accomplish and stimulate the process of converting petroleum reserves to methane but also no disclosure about a specific and rapid methodology to identify methanogens.

Methanogens are important members of microbiological consortia in natural environments, subterranean formations including petroleum reservoirs and also in marine and land animals, insects and human gut, peat bogs, waste streams, etc. However, there is no standard method of detecting methanogens. One method of methanogen detection is to culture them (2, 3, 4, 5, 6, 7, 8, 9). Cultivating methanogens anaerobically in a laboratory is a laborious and time-consuming process. Another method of identifying methanogens is to use rRNA-targeted archeabacteria specific PCR primers (10, 11) or methanogen specific group-specific 16s rDNA probes (12, 13). These methods suffer from a limitation wherein the probes cross-react with organisms of other physiological, or even phylogenetic groups when applied to environmental samples containing unknown sequences (1).

A. Role and Genes of Methyl Reductase

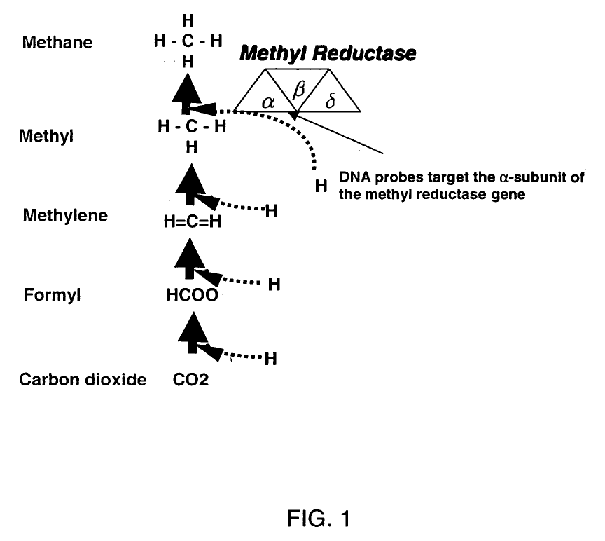

Methanogens belong to Archaea group of microorganisms and are classified into three major groups namely, Methanomicrobiales, Methanobacteriales, and Methanococcales. All methanogenic bacteria employ elements of the same biochemistry to synthesize methane. Methanogenesis is accomplished by a series of chemical reactions catalyzed by metal-containing enzymes that reduce CO₂ to CH₄ by adding one hydrogen atom at a time (FIG. 1). Methyl reductase catalyses the conversion of methyl-coenzyme COM to methane in the terminal step of methanogenesis in methane bacteria. The presence of methyl reductase is common to all diverse methanogens, and therefore it is the definitive and characteristic feature of methanogenic bacteria and unique only to them (14).

B. Design of the Methanogen Specific DNA Probes

The methyl reductase enzyme is comprised of three proteins, labeled the α, β, and δ subunits (15), which are encoded for in the DNA of methanogens by three methyl reductase genes, mcrA, mcrB and mcrG, respectively and the enzyme itself has been isolated from a number of methanogens (16, 17, 18, 19, 20). Specifically, the genes encoding the mcrA have been cloned and sequenced from several methane bacteria (16, 17, 21, 22, 23, 24). The methyl reductase protein sequences were deduced by translating the DNA sequence into amino acid sequence. The reasons for translating genetic codes into protein sequences are two-fold. First, the genetic code is the blue print used to produce the functioning enzyme and it is the functional form of the protein that is under evolutionary pressures. Second, the genetic code is redundant in that more than one genetic code can produce the same enzyme. Therefore, in designing primers for PCR it is desirable to obtain the least degenerate universal primers that specifically targets all methyl reductase genes.

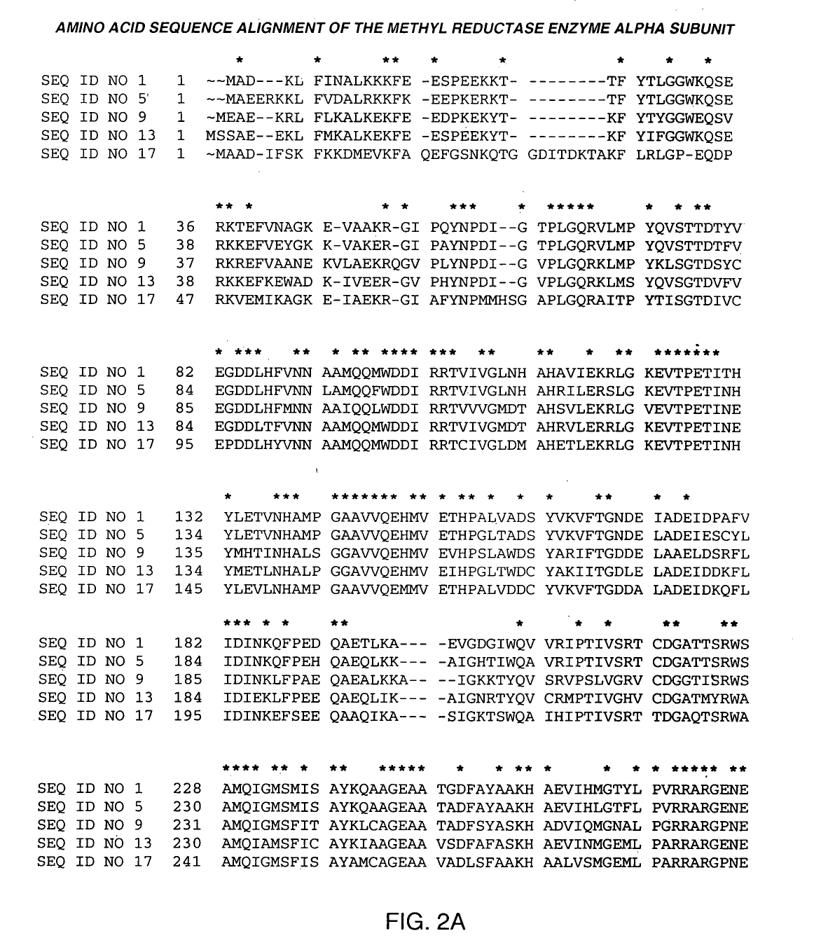

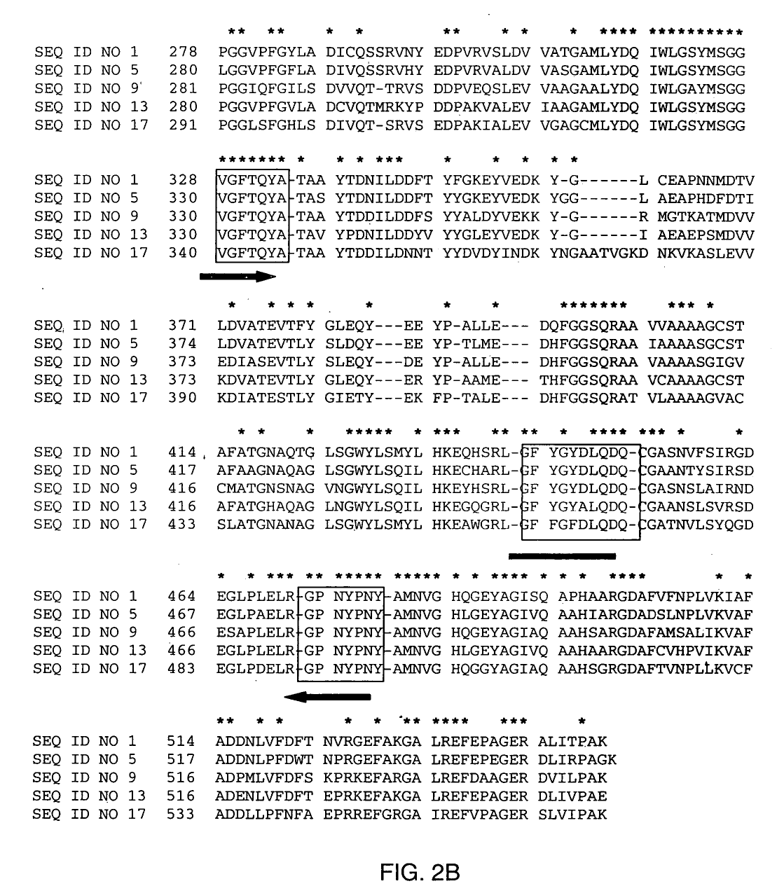

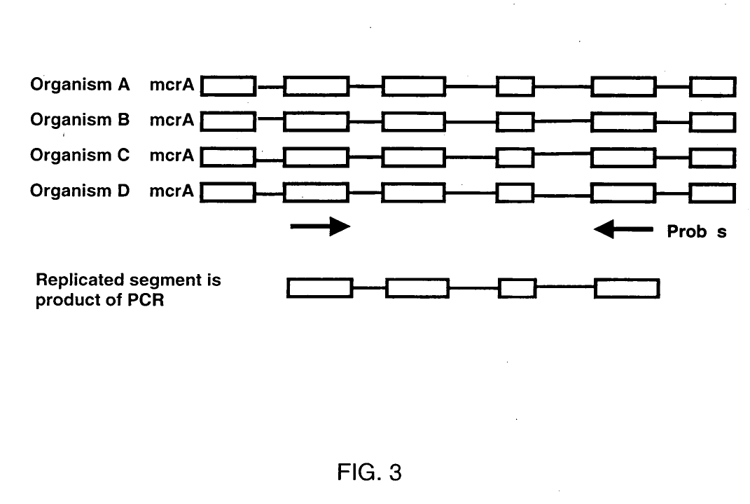

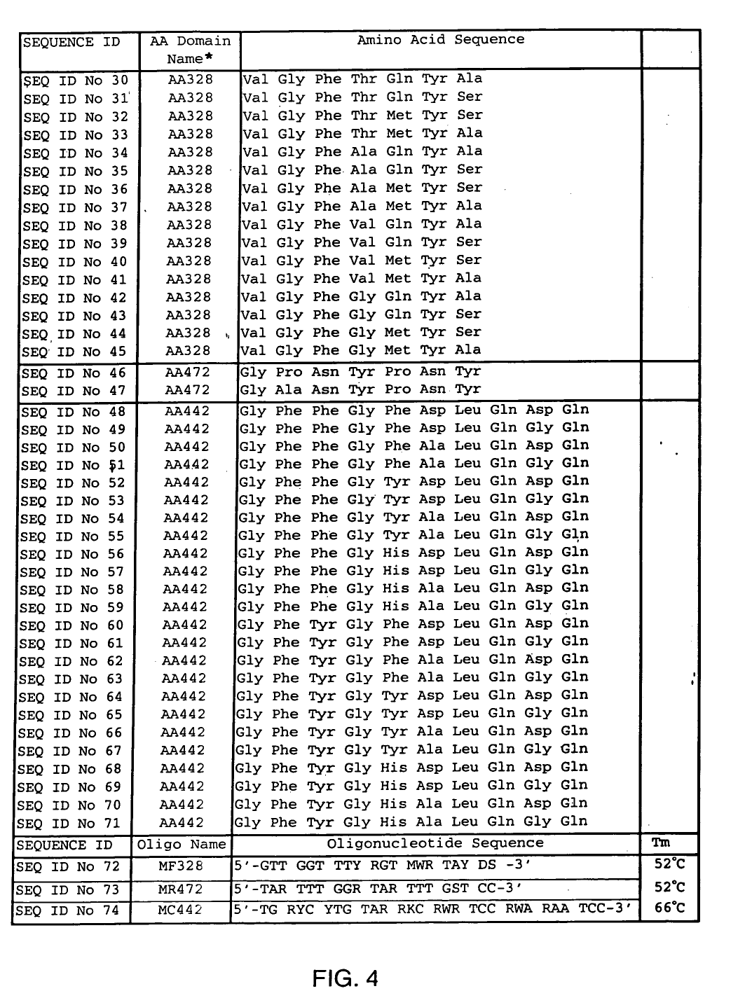

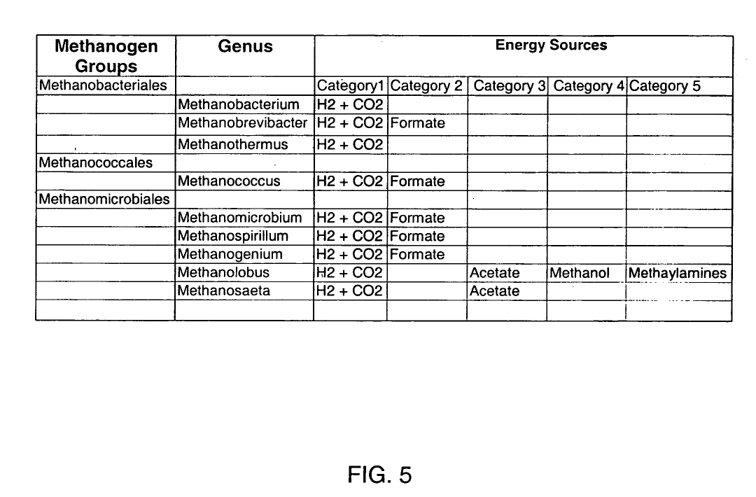

A comparison by alignment of the amino acid sequence of the methyl reductase alpha subunits from various methanogens reveals several segments in the protein sequences that are identical or have amino acid substitutions with similar physical properties. The detailed list of mcrA amino acid sequences and the segments with identical or similar physical properties within each protein sequence is given in SEQ ID NOs. 1 through 29. An alignment of SEQ ID NOs. 1, 5, 9, 13 and 17, where single letter amino acid codes have been used in conformity with widely practiced procedure in the art (32), illustrates such segments in mcrA protein sequences. (FIG. 2). A line diagram represents an alignment of simplified version of methyl reductase gene sequences (FIG. 3). These regions are conserved because they form the actual chemical structures required for the enzyme's function; hence they are called structure-function domains. These conserved regions have the least codon degeneracy for the following reasons; identical amino acids at a position reduces the multiplicity, and at an alignment position with amino acids with similar physical properties frequently have similar condon usage. The least codon degeneracy in conserved structure-function domains is an advantage in reducing the complexity of the primer or probe nucleotide sequences. Previously, PCR primers based on a limited set of mcrA genes or gene sequences derived from respective mcrA protein sequences were used to specifically identify methanogens (25, 26, 27). These initial efforts in detecting methanogens based on methyl reductase genes also made available a number of sequences of other methyl reductase proteins or the encoding genes. It is well known in the art that the availability of number of methyl reductase protein sequences or the DNA sequences of genes encoding methyl reductase influence the confidence and accuracy in the design of PCR primers capable of targeting all methyl reductase genes in all methanogens. The variability in methyl reductase protein sequences arises from the adaptation of methanogens to different environments and available nutrition. For instance, the different metabolic requirements some of the methanogens is illustrated as an example (FIG. 5). Therefore, for this invention, we have retrieved, as of this date, all available, partial or full length; mcrA sequences from a public database (Genbank database at the National Center for Biotechnology Information) in designing universal methanogen specific PCR primers. In reducing this invention to practice three conserved structure-function amino acid domains, namely, AA328 (SEQ ID Nos 30 through 45), AA472 (SEQ ID Nos 46 and 47) and AA442 (SEQ ID Nos 48 through 71) were identified for PCR probing. These amino acid sequences, as represented in FIG. 4, were used in deducing the nucleic acid sequences, namely, MF328 (SEQ ID No 72), and MR472 (SEQ ID No 73), as the PCR primers and MC442 (SEQ ID No 74), as the internal probe (FIG. 4). The polynucleotide sequences of the invention further include all the degenerate sequences as set forth in SEQ ID NOs 72, 73 and 74. In a preferred embodiment, the polynucleotide sequences of the invention comprise of at least ten sequential nucleotides, more preferably 20 sequential nucleotides in length and still more preferably 40 nucleotides in length.

According to one aspect of this invention, our DNA probes, as set forth in SEQ ID No 72 (MF328), and SEQ ID No 73 (MR472) in FIG. 4, detect two unique key structure-function domains and not the entire complex of mcrA genes (FIG. 3). The two conserved stretches of methyl reductase amino acid sequences are unique and found only in that enzyme and have not been the target of probing mcrA genes in other studies (25, 26, 27). The conserved domain that formed the basis for designing MF328 is represented by AA328 and that of MR472 is represented by AA472 respectively in FIG. 4. The presence of both conserved sequences is required for a positive test of probing and exponential amplification of mcrA DNA. Probing both sites simultaneously with appropriate experimental controls provides a high level of confidence (>99.9%) for a positive test for the presence of methanogens. In addition, the DNA sequence of the methyl reductase detected will be determined and compared to known enzyme sequences to verify our results and infer the identity of the organism that produced the mcrA gene.

A cursory examination of the alignment of mcrA sequences in FIG. 2 clearly identifies the three conserved regions, as represented by boxes, having similar or identical amino acid residues but the position of each amino acid residue varies within a given mcrA sequence. Therefore, the sequences identified in FIG. 4 follow a naming convention wherein each conserved region is designated by a name that comprises of a two-letter code and a three-digit number. For instance, mcrA forward primer sequence is described as MF328. “M” stands for the gene mcrA, “F” stands for forward primer and the number, “328” refers to the amino acid position in the sequence constituting methyl coenzyme M reductase I alpha subunit of Methanobacterium thermoautotrophicum. The Genbank database accession number for this sequence is AAA73445 (24) and the sequence is depicted in FIG. 2. Similarly, the letters “R” and “C” refer to “reverse” and “central” in MR472 and MC442 respectively. In FIG. 4, ‘AA’ refers to amino acid.

B1. Internal Probe

According to another aspect of the invention a third probe, that is internal to the two probes described previously, targets yet another structure-function domain of the mcrA gene and its DNA sequence is as set forth fully in SEQ ID No 74 (MC442) in FIG. 4. The conserved amino acid domain used in the design of MC442 is represented by AA442 in FIG. 4. While reducing the present invention to practice, this probe, or its complement, may be used in place of one of the two PCR probes described previously in a PCR amplification process, or singly in a primer extension or probe hybridization assays. However, this probe, is intended but not limited to, for application in nucleic acid quantification or detection assays such as 5′ to 3′ nuclease assay (U.S. Pat. Nos. 5,210,015, 5,487,972, etc.). Alternatively, the probe can be used in improved techniques that employ modification with fluorescent reporter and quencher dyes at the 5″and 3″ end respectively (28) in conjunction with the two PCR primers, MF328 and MR472, described previously in FIG. 4.

Specifically, the probe is used in the determination of number of methanogens using quantitative PCR or in monitoring amplification of nucleic acid targets in real time by techniques such as DNA sequence detection system (Applied Biosystems, CA) or other techniques known to those skilled in the art. The determination of methanogens type, species identification by mcrA gene amplification, cloning, DNA sequencing and comparative DNA analyses, and in determining sample contamination, and analysis of forensic samples are also within the scope of this invention.

Standard DNA manipulation techniques such as cloning, plasmid DNA purification and DNA sequencing are routine in the art or ready-to-use kits for many of these manipulations are commercially available. Even so, laboratory procedures for these and other techniques are usually found in standard manuals for molecular biology protocols (29). Several other references and procedures are provided throughout this document for the convenience of the reader, although they are well known in the art.

C. Microbial DNA Isolation

The first step in DNA technology is the isolation of the genetic material from cells. The DNA isolation procedure we devised uses enzymes, detergents and heat treatments to remove the skin or the outer shell of microorganisms releasing their DNA into solution. The DNA solution is a complex mixture of genetic material from all of the microorganisms that were in the reservoir sample.

In a preferred embodiment, nucleic acids, such as DNA and RNA are extracted from the sample and are analyzed in their extracted form. Methods of extracting nucleic acids from nucleic-acid containing samples are well known in the art (30, 31). One such, non-limiting, method is further described below. Additional methods are described, for example, in standard protocols, which is incorporated by reference (29).

C1. Details of DNA Isolation

The application of DNA technology for the analysis of natural microbial populations depends on the ability to extract high molecular weight DNA from every organism in an environmental sample. This protocol is a description for the lysis of microorganisms in sediments and in liquids, which is based on a series of enzymes, detergents and heat and obtain a crude extract of nucleic acids by salting-out from the cell lysate.
1. Place 8 g of sediment in a 35 mL polypropylene screw-cap centrifuge tube and resuspend in 20 mL of 0.3% w/v sodium pyrophosphate solution containing 2% w/v polyvinylpyrrolidone (PVP). Shake the sediment slurry at 150 rpm for 1 hour. Centrifuge at 20,000×g for 10 minutes at 4° C. Note: Each environmental sample is unique, which requires certain steps to be modified. It is important to centrifuge hard and long enough to pellet derbies; therefore the g-forces used for centrifugation depend upon the properties of the sample. Perform additional washes for 15 minutes shaking on the tilt-rocker until the supernatant appears clarified upon centrifugation. For example, marine oil-seep sediments require a total of 5 washes while soil may require 2 washes. In the case of water samples, repeated centrifugation is resorted to, to recover adequate biomass or it is filtered to harvest the biomass on the filter and the DNA extracted.
2. Resuspend the washed-sediment in 20 mL or approximately 2.5 t 3.0 times its own volume of bacterial lysis buffer (50 mM Tris-HCl pH 8.0, 25 mM EDTA pH 8.0, 0.3M sucrose, 2% w/v PVP and 5 mg/mL lysozyme) by vortexing. Place on tilt-rocker for 15 minutes to 1 hour at room temperature.
3. Add 2.0 mL or proteinase K (20 mg/mL in 50 mM Tris-HCl, pH 8.0, and 25 mM EDTA, pH 8.0) and 2.0 mL 20% w/v sodium dodecyl sulfate. Incubate for 30 minutes at room temperature on the tilt-rocker.
4. Lyse or break the cells by adding 2 mL of 5M NaCl and 2 mL of 10% w/v CTAB (hexadecyltrimethyl ammonium bromide) in 0.7 M NaCl solution and incubating at 65° C. for 30 minutes.
5. Purify the genomic DNA by extracting with a volume approximately equal to the slurry, which is typically 15 mL (25:24:1, v/v/v) phenol/chloroform/isoamylalcohol, mix by gently inverting.
6. Separate the aqueous and the organic phases, and pellet the debris by centrifugation at 10,000×g for 10 minutes at 4° C.
7. Carefully draw-off the supernatant and place in a clean polypropylene tube and precipitate DNA by adding 0.6 volumes of isopropanol and incubating overnight at −20° C. Pellet the DNA by centrifugation at 20,000×g for 20 minutes.
   D. Polymerase Chain Reaction (PCR)

Each microorganism's DNA contains approximately 2,000 or more genes, which emphasizes the technical challenge to be able to discriminate a methanogen gene, mcrA, the alpha subunit of methyl reductase, from thousands of other genes. The environmental sample being analyzed for the presence of methanogens, according to the present invention, can be diluted prior to the nucleic acid based analysis as described herein. Typically, there is not a sufficient amount of DNA material isolated from reservoir bacteria to effectively test for individual DNA sequences directly. The limited amount of DNA material further complicates detecting a single gene in a complex mixture. To overcome these technical hurdles, a technique called the polymerase chain reaction is performed.

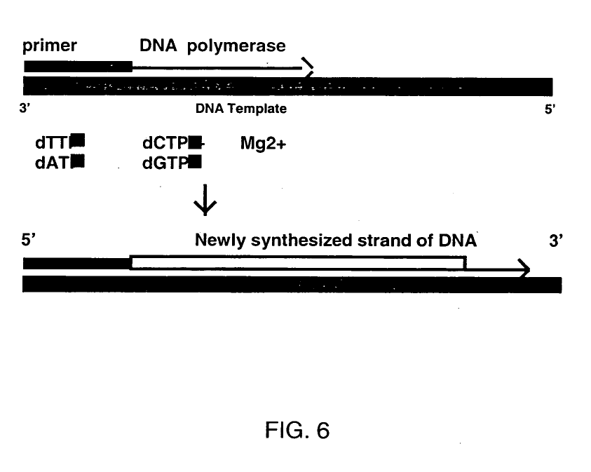

The polymerase chain reaction (PCR) is the repetitive synthesis of a targeted region of DNA accomplished by mimicking the natural process of DNA replication (FIG. 6). The specific replication of the gene coding for methyl reductase DNA is achieved by using two key structure-function domains from the mcrA gene to promote DNA synthesis (FIG. 3, highlighted by the arrows). If a mcrA gene or multiple mcrA genes from different organisms are present, only then, will their numbers be amplified a million-fold, yielding a sufficient quantity for analysis. The products of the PCR reaction are mcrA gene fragments containing both probe sequences at their respective ends and the intervening sequence. Therefore, the PCR amplification of the methyl reductase gene in a biological sample, including but not limited to petroleum reservoir bacterial DNA, indicates the presence of methanogens. The unique sequence in the intervening region can be used to identify which methanogen produced the mcrA genes by comparative sequence analysis.

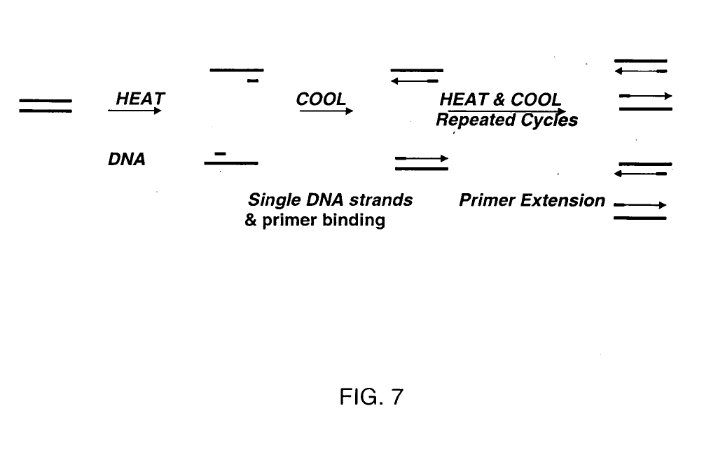

The polymerase chain reaction employs two short fragments of DNA, called primers, each complementary to the opposite strands of the region of DNA to be amplified. The primers are arranged so that each primer extension reaction directs the synthesis of DNA towards the other. The amplification process is initiated by separating the two strands of DNA by heating to allow for the respective primers to bind to their complementary single strand of DNA. The reaction is cooled to activate the DNA polymerase, which use both primers as sites for initiating DNA synthesis by the extension reaction. The heating and cooling cycle is typically repeated 30 to 40 times and the DNA accumulates exponentially until millions of copies are synthesized (FIG. 7).

D1. Details of PCR

Template: 1 μL of the undiluted genomic DNA or its dilution by 10 or 20 times
Water: 72.75 μL
Primer 1: 10 picomoles of MF328, SEQ ID No. 72
Primer 2: 10 picomoles of MR472, SEQ ID No. 73
dNTPs: 8 μL of 200 uM stock
MgCl₂: 4 μL of 25 mM stock
Taq polymerase: 0.25 μL
PCR buffer: 10 μL of 10× stock
Total volume: 100 μL

The PCR was performed in a Perkin-Elmer 9600 GeneAmp PCR machine as follows.
94° C. for 2 minutes for initial denaturation of the genomic DNA followed by 30 or 40 cycles of
92° C. for 30 seconds to denature the genomic DNA
50° C. for 30 seconds to anneal the primers
72° C. for 90 seconds to extend the annealed primers
   following which the sample was held at 72° C. for 10 minutes and cooled to 4° C. until use. When practicing the present invention the reaction parameters are suitably modified and the reaction itself may be repeated where the degree of specificity or efficiency of amplification reaction is considered insufficient. Such modifications may involve changes in temperature, time, thermal cyclers, reagents or their concentration, etc.

The amplification products resulting from a polymerase chain reaction were separated and visually detected using dye-based agarose gel electrophoresis and their size determined by comparing them with appropriate DNA molecular ladders. Other suitable size determination techniques for analyzing the PCR amplified products include capillary electrophoresis and automated fluorescence DNA analyzers such as those used in automated DNA sequencing and genotyping and several hybidization and mass spectroscopy formats. These latter methods are especially useful for the detection of amplified nucleic acid product where a labeled nucleotide is incorporated into the amplified strand by using labeled primers. Primers employed in the PCR process have been labeled with radioactivity, biotin, fluorescent dyes, digoxygenin, horseradish peroxidase, alkaline phosphatase and acridinium esters.

E. DNA Sequence Identification of Methanogens

PCR amplification of DNA extracted from reservoir bacteria is a presumptive test for the presence of methanogens. In order to rule out a “false-positive” result, the DNA sequence of the amplified DNA fragment(s) is confirmed as a mcrA gene sequence by nucleotide sequence analysis. DNA sequence analysis involves several steps: cloning to isolate individual DNA fragments; DNA sequencing isolated DNA fragments; DNA sequence similarity search of a nucleotide database; and finally, comparative sequence analysis.

E1. Purification of PCR product

The reaction mixture may have unreacted primers, excess dNTPs, primer-dimers, etc., which may affect the cloning efficiency. In order to increase the likelihood of obtaining recombinant DNA with the nucleic acid molecules of interest, that is mcrA PCR product, it is important to purify the PCR reaction mixtures. The PCR reaction mixtures are purified using any of the commercially available kits and the purified product used in the cloning process.

E2. Cloning

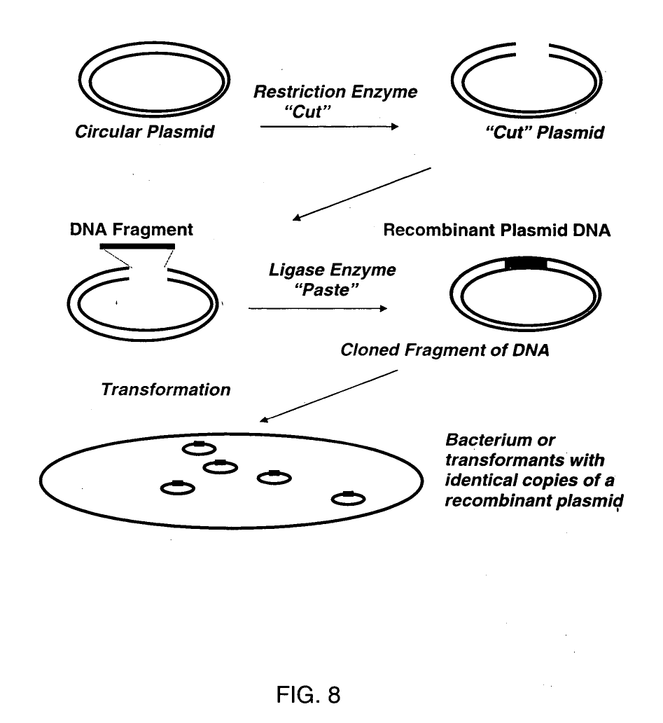

The products of the PCR reaction are fragments of the mcrA gene(s) that have to be individually isolated before the DNA sequence of each unique gene fragment can be determined (FIG. 8). Cloning is the procedure used to isolate and purify individual mcrA DNA fragments in the mixture of PCR reaction products. The cloning procedure integrates a specific DNA fragment into a replicating genetic element, such as a plasmid, so that it can be isolated and replicated in a bacterium. Plasmids are small, circular DNA molecules that occur naturally in bacteria where they replicate independently. They are ideal for cloning because they are small and can “recombine” foreign genes or fragments of DNA. Enzymes are used to graft into or excise fragments of DNA from plasmids. A circular plasmid is either cut at a single site by a restriction enzyme or such cut plasmids are obtained from commercial suppliers and the foreign fragment of DNA is inserted in that opening which reforms a circle mediated by another enzyme called ligase. This procedure is analogous to “cut and paste” and referred to as cloning. The hybrid or grafted plasmid, called recombinant DNA, is reintroduced into bacteria. When the plasmids are mixed with bacteria that are able to take up DNA, only a single plasmid is admitted into each cell. As these bacteria grow on the solid medium the plasmid replicates inside as each cell repeatedly divides and produce individual colony generating enormous numbers of copies of the original DNA fragment. Each bacterial colony is comprised of many bacteria containing the identical rDNA therefore they are called clones. In order to identify the microorganisms that produce a mcrA gene, dozens of bacterial clones from a library constructed from reservoir DNA will be DNA sequenced.

E3. rDNA Purification

The plasmids carrying PCR amplified DNA fragments of mcrA gene are isolated from several bacterial clones in preparation for DNA sequencing. Standard procedures are well known in the art for the purification of the rDNA molecules from bacterial colonies (29).

E4. Automated DNA Sequencing

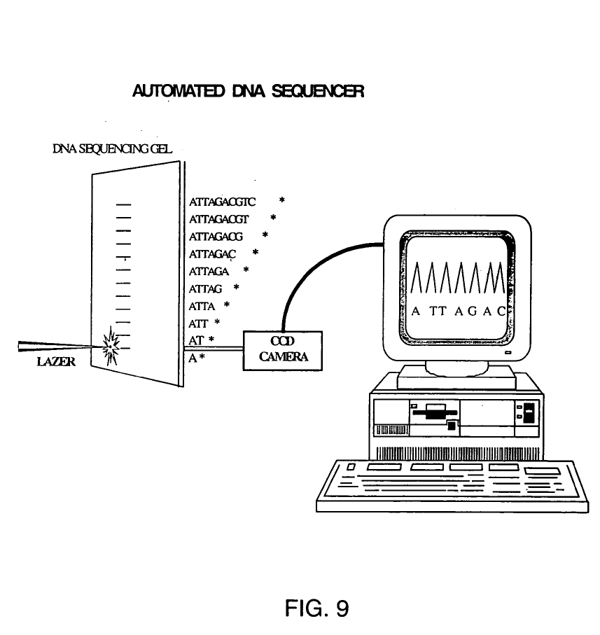

The essential elements of automated DNA sequencing are shown in FIG. 9. The determination of the sequence of nucleotides in a fragment of mcrA DNA requires a step that uses one stand of the double helix as a template to generate partial fragments of itself. Each partial DNA fragment is terminated with a fluorescent labeled nucleotide (each nucleotide is labeled with a different fluorescent dye) that is used to identify which one of the four nucleotides is at the end of the DNA strand. The mixture of labeled DNA strands are placed on the top of the DNA sequencing gel then forced through the gel matrix by an electric field. The polyacrylamide gel electrophoresis process separates the DNA strands according to their length with the smallest strand leading followed by DNA strands progressively one nucleotide larger in length. A laser is used to excite each fluorescent nucleotide as it passes by the detector identifying the terminal nucleotide. The computer displays these events as peaks of fluorescence and assigns the identity of the terminal nucleotide sequentially building the mcrA DNA sequence. Commercially available DNA sequencing kits are used in accordance with the protocol suggested by the supplier.

F. Detection and Identification of Methanogens

F1. Similarity Search of Databases

Database similarity searching is used to determine which of the many sequences present in the databases are potentially related to the sequence derived from reservoir DNA. Sequence similarity is expressed as a score based upon percent identity, but it does not determine whether these sequences display sufficient similarity to justify any inference to a common ancestry or function. What it does provide is an evaluation of the sequence to justify or not for further comparative analyses. Comparative sequence analysis is required to determine if sequences are homologous, meaning they have a common ancestral origin and function. Computer software has been written to automate the extraction and reformatting of raw data produced by the DNA sequencer, and to electronically send to an external server for an extensive database similarity search. The data in the electronic reply to our query is extracted and routed to a folder created for each project. The automation of data analysis greatly enhances our productivity.

F2. Comparative Sequence Analysis

Comparative analysis has a long history in biology, i.e., Darwin's comparison of morphological features provided the foundation for the theory of natural selection. The tradition continues but in much greater detail —at the level of individual DNA bases or amino acids. Once a sequence with sufficient similarity to mcrA is identified, the first step in extracting information from the molecular sequence is to compare it with other mcrA sequences (FIG. 2 is an example). Comparison of DNA or protein (amino acid) sequences requires an alignment of the sequences, which is an explicit mapping between residues of two or more sequences. The objective in aligning sequences is to place all of the ‘homologous’ positions in correspondence with one another. Here homology means much more than similarity or likeness; it signifies “retention of ancestral attributes” which preserves a structure with a specific function. Therefore, it is an alignment of homologous structures that have a common function, which must be emphasized, because it is the functional form of the molecule that is under evolutionary pressures. Please note, the mcrA DNA sequence that was determined from reservoir bacterial DNA was also translated into the amino acid sequence, because it is the protein that forms the functional enzyme. Difference in amino acid sequence between the mcrA genes is the culmination of a single or an unknown series of mutational events. Mutational events can result in a simple change in the type of amino acid, or alter the length of the molecule by addition (insertions) or removal (deletions) of the mcrA DNA. Insertion or deletion of amino acids requires the introduction of “alignment gaps” or “indels” (represented by hyphens, -) to align homologous regions of sequences with different lengths.

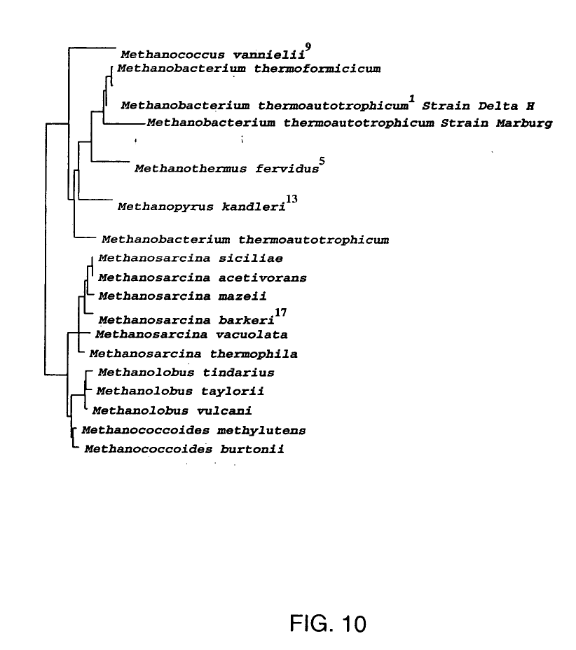

Two types of statistical analysis are typically used to infer evolutionary or phylogenetic relationships from aligned molecular sequences: evolutionary distance and maximum parsimony methods. The distance methods only look at the quantitative difference, the number of positions that differ between each pair of sequences, which is used as a measure of evolutionary distance. The parsimony methods are more of a qualitative measure, i.e., it considers if the positions differ and the nature of the differences. Simply stated, distance analysis is a simple measure of pair-wise differences, while the parsimony analysis attempts to reconstruct the history of the changes. The results of phylogenetic analyses are represented as a “tree”, a pictogram that helps visualize the historical relationships and assist in determining which microorganism produced the mcrA gene (FIG. 10). Such trees are built using the amino acid sequence in the alpha subunit of methyl reductase. The name of the organisms that produced the proteins are color coded highlighting the three major groups of methanogens, Methanococcales, Methanobacteriales, and Methanomicrobiales.

G. Definitions

In one aspect of the invention, the nucleic acid sample to be assayed is obtained from a biological sample that is a solid like soil, sediment or ditch mud or aqueous liquids like lake water, petroleum formation waters, etc. The term “sample” is used in its broadest sense. A sample suspected of containing a nucleic acid can comprise a cell or cellular contents such as its DNA, RNA, plasmid DNA, etc. In another aspect of the method, the predetermined nucleic acid target sequence is present in the sample for the purpose of gene modification.

A “nucleic acid,” as used herein, is a covalently linked sequence of nucleotides in which a phosphodiester group links the 3′ position of pentose of one nucloetide to the 5′ position of pentose of the next nucleotide. The nucleotide residues (bases) are linked in specific sequence, i.e., a linear order of nucleotides. A “polynucleotide,” as used herein, is a nucleic acid containing a sequence that is greater than about 100 nucleotides in length. An “oligonucleotide,” as used herein, is a short polynucleotide or a portion of a polynucleotide. An oligonucleotide typically contains a sequence of about two to about one hundred bases. The word “oligo” is sometimes used in place of the word “oligonucleotide”.

In referring to “isolated nucleic acid molecule(s)” it is intended to be a nucleic acid molecule, either DNA or RNA, that has been removed from its native environment. For instance, DNA removed from a microbial cell or recombinant DNA molecules contained in a plasmid DNA are considered isolated for the purposes of the present invention. Other examples of isolated DNA molecules include recombinant DNA molecules maintained in heterologous host cells, purified (partially or substantially) DNA molecules in solution, and synthetic nucleic acid molecules. In vitro or in vivo RNA transcripts of the DNA molecules of the present invention are also considered as isolated nucleic acid molecules. Isolated nucleic acid molecules of the present invention include, but are not limited to, single stranded and double stranded DNA, and single stranded RNA, and complements thereof. Isolated nucleic acid molecules of the present invention include DNA molecules having a nucleotide sequence substantially different than the one describing, for instance, methyl reductase in FIG. 2, but which, due to the degeneracy of the genetic code, still encode a methyl reductase protein. The genetic code is well known in the art and degenerate variants are routinely generated.

A “nucleic acid of interest,” as used herein, is any particular nucleic acid one desires to study in a sample.

A base “position” as used herein refers to the location of a given base or nucleotide residue within a nucleic acid.

As used herein, the term “target nucleic acid” or “nucleic acid target” refers to a particular nucleic acid sequence of interest. Thus, the “target” can exist in the presence of other nucleic acid molecules or within a larger nucleic acid molecule.

As used herein, the term “nucleic acid probe” refers to an oligo-nucleotide or polynucleotide that is capable of hybridizing to another nucleic acid of interest. A nucleic acid probe may occur naturally as in a purified restriction digest or be produced synthetically, recombinantly or by PCR amplification. As used herein, the term “nucleic acid probe” refers to the oligonucleotide or polynucleotide used in a method of the present invention. That same oligonucleotide could also be used, for example, in a PCR method as a primer for polymerization, but as used herein, that oligonucleotide would then be referred to as a “primer”. Herein, oligonucleotides or polynucleotides may contain a modified linkage such as a phosphorothioate bond.

As used herein, the terms “complementary” or “complement” are used in reference to nucleic acids base pairing rules (i.e., a sequence of nucleotides). The base-pairing rules are well known in the art, where A pairs with T and C pairs with G. For example, the sequence 5′-T-A-C-Y 3′, is complementary to the sequences 3′-A-T-G-C-5′ or 3′-A-T-G-T 5′. The term “degenerate” means that the letter codes other than A, G, T and C in the genetic code are employed to designate variable nucleotides at the same position in a given sequence. For instance, S may mean a G or a C, W may mean an A or a T, Y may mean a C or a T, K may mean a T or a G, M may mean an A or a C, D may mean an A or a G or a T and R may mean an A or a G.

When only some of the nucleic acid bases are matched between two strands of nucleic acid according to the base pairing rules complementarity can be “partial”. Complementarily between the nucleic acid strands may be “complete” or “total” when all of the bases are matched. The extent of complementarity between nucleic acid strands has significant effects on the efficiency and strength of hybridization between nucleic acid strands. Extent of complementarity is of particular importance in detection methods that depend upon binding between nucleic acids, such as those of this invention. When a probe hybridizes to either or both strands of the target nucleic acid sequence under defined conditions of stringency the term “substantially complementary” may be employed. As applied to any primer extension reactions, the nucleic acid probe or primer is referred to as partially or totally complementary to the target nucleic acid that refers to the 3′ terminal region of the probe (i.e., within about 10 nucleotides of the 3′ terminal nucleotide position).

Degree of complementarity is often described in terms of “homology” between two nucleic acid molecule. Homology (identity) can be partial or complete. A nucleic acid sequence that is partially complementary is said to be “substantially homologous” when it partially inhibits a completely complementary sequence from hybridizing to a target nucleic acid.

The term “substantially homologous,” as applied to a double-stranded nucleic acid sequence such as a cDNA or genomic clone or a single-stranded nucleic acid template sequence, refers to a probe that can hybridize to a strand of the double-stranded nucleic acid sequence under conditions of low stringency.

The temperature at which 50% of a population of double-stranded nucleic acid molecules becomes dissociated into single strands is referred to as the “melting temperature” (Tm). The equations for calculating the Tm of nucleic acids are well known in the art. One such equation for estimating Tm of an oligo is given by the formula 2(A+T)+4(G+C). Other more sophisticated formulae for the computation of Tm exist in the art, which take into account the structural as well as sequence characteristics of a primer. A computed Tm is merely an estimate and the optimum temperature is commonly determined empirically. Usually, the primer annealing temperature is 2° C. to 5° C. below the Tm of that primer. The nucleic acid probe is designed not to hybridize with itself to form a hairpin structure in such a way as to interfere with hybridization of the 3′-terminal region of the probe to the target nucleic acid. Parameters guiding probe design are well known in the art. Commercially available software for designing PCR primers can also be used to assist in the design of probes for use in the invention.

The term “hybridization” refers to the base pairing between complementary nucleic acid strands. Many factors affect hybridization and the strength of hybridization (i.e., the strength of the association between nucleic acid strands). These factors include, among others, the degree of complementarity between the nucleic acids and the stringency of hybridization. The latter factor is, in turn, influenced by such conditions as the concentration of salts, the Tm (melting temperature) of the nucleic acid hybrid, the presence of other components (e.g., the presence or absence of polyethylene glycol), the molarity of the hybridizing strands and the G:C content of the nucleic acid strands.

The term “stringency” is used in reference to the conditions of temperature, ionic strength, and the presence of other compounds, under which nucleic acid hybridizations are performed. Under “high stringency” conditions, nucleic acid base pairing will occur only between nucleic acid fragments that have a high frequency of complementary base sequences. Thus, conditions of “weak” or “low” stringency are often required when it is desired that nucleic acids that are not completely complementary to one another to be hybridized or annealed together. The art knows well that numerous equivalent conditions can be employed to comprise low stringency conditions.

The terms “purified” and/or “to purify,” mean the result of any process, which removes some contaminants from the component of interest, such as a protein or nucleic acid. The percent of a purified component is thereby increased in the sample.

### H. REFERENCES

1. Holmes A J, Owens N J, Murrell J C. 1995. Detection of novel marine methanotrophs using phylogenetic and functional gene probes after methane enrichment. Microbiology. August;141 (Pt 8):1947-55.
2. Cox D J, Herbert S D. 1978. An anaerobic glove box for the isolation and cultivation of methanogenic bacteria. J. Appl. Bacteriol. December;45(3):411-5.
3. Perez-Fillol M, Rodriguez-Valera F, Ferry J G. 1985. Isolation of methanogenic bacteria able to grow in high salt concentration. Microbiologia. September;1(1-2):29-33.
4. Foster M S, Rodabough A, Dayton T M, Melko E M, Szegedi S S, Niederhoffer E C. 1993. Improved methods for the cultivation of strictly anaerobic, extremely thermophilic methanogens. Biotechniques. December; 15(6):996-8, 1000, 1002.
5. Pennings J L, Keltjens J T, Vogels G D. 1998. Isolation and characterization of Methanobacterium thermoautotrophicum DeltaH mutants unable to grow under hydrogen-deprived conditions. J. Bacteriol. May; 180(10):2676-81.
6. Ng T K, Weimer P J abd Gawel L J. 1989. Possible nonanthropogenic origin of two methanogenic isolates from oil producing wells in the San Miguelito filed, Ventura county, California. Geomicrobiology Journal. 7:185-192.
7. Nilsen R K and Torsvik, T. 1996. Methanococcus thermolithotrophicus isolated from North Sea oil field Reservoir water. Appl Environ. Microbiol. February;62(2):728-731.
8. Ollivier B, Cayol J. -L, Patel, B K C, Magot M, Fardeau M. -L and Garcia J. -L. 1997. Methanoplanus petrolearius sp. nov., a novel methanogenic bacterium from an oil producing well. FEMS Microbiol. Lett. 147:51-56.
9. Ollivier B, Fardeau M. -L, Cayol J. -L, Magot M, Patel, B K C, Prensier, G and Garcia J. -L. 1998. Methanocalculus halotolerans gen. nov., sp. nov., isolated from an oil producing well. Int J. Syst. Bacteriol. 48:821-828.
10. Zepp Falz K, Holliger C, Grosskopf R, Liesack W, Nozhevnikova A N, Muller B, Wehrli B, Hahn D. 1999. Vertical distribution of methanogens in the anoxic sediment of Rotsee (Switzerland). Appl. Environ. Microbiol. June; 65(6): 2402-8.
11. Munson M A, Nedwell D B, Embley T M. 1997. Phylogenetic diversity of Archaea in sediment samples from a coastal salt marsh. Appl. Environ. Microbiol. December; 63(12):4729-33.
12. Raskin L, Stromley J M, Rittmann B E, Stahl D A. 1994. Group-specific 16S rRNA hybridization probes to describe natural communities of methanogens. Appl. Environ. Microbiol. April; 60(4): 1232-40.
13. Raskin L, Zheng D, Griffin M E, Stroot P G, Misra P. 1995. Characterization of microbial communities in anaerobic bioreactors using molecular probes. Antonie Van Leeuwenhoek. November; 68(4): 297-308.
14. Ferry J G. 1992. Biochemistry of methanogenesis. Crit. Rev. Biochem. Mol. Biol. 27(6): 473-503.
15. Ellefson W L, Wolfe R S. 1981. Component C of the methylreductase system of Methanobacterium. J Biol. Chem. May 10; 256(9): 4259-62.
16. Bokranz M, Klein A. 1987. Nucleotide sequence of the methyl coenzyme M reductase gene cluster from Methanosarcina barkeri. Nucleic Acids Res. May 26; 15(10): 4350-1.
17. Bokranz M, Baumner G, Allmansberger R, Ankel-Fuchs D, Klein A. 1988. Cloning and characterization of the methyl coenzyme M reductase genes from Methanobacterium thermoautotrophicum. J. Bacteriol. February; 170(2): 568-77.
18. Allmansberger R, Bokranz M, Krockel L, Schallenberg J, Klein A. 1989. Conserved gene structures and expression signals in methanogenic archaebacteria. Can. J Microbiol. January; 35(1): 52-7.
19. Nolling J, Elfner A, Palmer J R, Steigerwald V J, Pihl T D, Lake J A, Reeve J N. 1996. Phylogeny of Methanopyrus kandleri based on methyl-Coenzyme M reductase operons. Int. J Syst. Bacteriol. October; 46(4): 1170-3.
20. Grabarse W, Mahlert F, Shima S, Thauer R K, Ermler U. 2000. Comparison of three methyl-coenzyme M reductases from phylogenetically distant organisms: unusual amino acid modification, conservation and adaptation. J. Mol. Biol. October 20; 303(2): 329-44.
21. Weil C F, Sherf B A, Reeve J N. 1989. A comparison of the methyl reductase genes and gene products. Can. J Microbiol. January; 35(1): 101-8.
22. Cram D S, Sherf B A, Libby R T, Mattaliano R J, Ramachandran K L, Reeve J N. 1987. Structure and expression of the genes, mcrBDCGA which encode the subunits of component C of methyl reductase in Methanoccous vannielli. Proc. Natl. Acad. Sci. USA. 84:3992-3996.
23. Klein A, Allmansberger R, Bokranz M, Knaub S, Muller B, Muth. 1988. Comparative analysis of genes encoding methyl coenzyme M reductase in methanogenic bacteria. Mol. Gen. Genet. August; 213(2-3): 409-20.
24. Pihl T D, Sharma S, Reeve J N. 1994. Growth phase-dependent transcription of the genes that encode the two methyl coenzyme M reductase isoenzymes and N5-methyltetrahydromethanopterin:Coenzyme M methyltransferase in Methanobacterium thermoautotrophicum delta H. J Bacteriol. October; 176(20): 6384-91.
25. Springer E, Sachs M S, Woese C R, Boone D R. 1995. Partial gene sequences for the A subunit of methyl-coenzyme M reductase (mcrI) as a phylogenetic tool for the family Methanosarcinaceae. Int. J Syst. Bacteriol. July; 45(3): 554-9.
26. Hales B A, Edwards C, Ritchie D A, Hall G, Pickup R W, Saunders J R. 1996. Isolation and identification of methanogen-specific DNA from blanket bog peat by PCR amplification and sequence analysis. Appl. Environ. Microbiol. February; 62(2): 668-75.
27. McDonald I R, Upton M, Hall G, Pickup R W, Edwards C, Saunders J R, Ritchie D A, Murrell J C. 1999. Molecular Ecological Analysis of Methanogens and Methanotrophs in Blanket Bog Peat. Microb. Ecol. October; 38(3): 225-233.
28. Livak J K, Flood S J A, Marmaro J, Giust, w and Deetz K. 1995. Oligonucleotides with fluorescent dyes at opposite ends provide a quenched probe system useful for detecting PCR product and nucleic acid hybridization. PCR Methods Applic. 4:357-362.
29. Sambrook J, Fritsch E F and Maniatis T. Molecular Cloning—A Laboratory Manual, Cold Spring Harbor Laboratory, Cold Spring Harbor, N.Y. (1989).
30. Selenska S and Klingmuller W. 1991. DNA recovery and direct extraction of Tn5 sequences from Soil. Lett. Appl. Microbiol. 13:21-24.
31. Orphan V J, Taylor L T, Hafenbradl D, Delong E F. 2000. Culture-dependent and culture-independent characterization of microbial assemblages associated with high-temperature petroleum reservoirs. Appl. Environ. Microbiol. February; 66(2): 700-11.
32. Lehninger A L. The amino acid building blocks of proteins, p. 72. In Biochemistry, second edition. Worth Publishers Inc, New York, N.Y. (1978).

1. A nucleic acid probe for the detection of the presence and/or Amount of methanogens in terrestrial and subterranean formations comprising at least ten sequential nucleotides or the complement of the ten sequential nucleotides that encode for a segment of one of the amino acid sequences in one of the three groups, as set forth in SEQ ID NOs 30 through 45 (AA328), SEQ ID NOs 46 and 47 (AA472) or SEQ ID NOs 48 through 71 (AA442) of FIG. 4.

2. An isolated nucleic acid probe for the detection of the presence and/or amount of methanogens in terrestrial and subterranean formations comprising at least ten sequential nucleotides from one of the nucleotides selected from the group consisting of nucleotides as set forth in SEQ ID NO 72, SEQ ID NO 73 and SEQ ID NO 74.

3. The probe of claim 1 wherein said probe comprises said complement of the ten sequential nucleotides.

4. The probe of claim 1 wherein said probe comprises said ten sequential nucleotides.

5. The probe of claim 2 wherein said nucleotide sequence is at least 90% homologous to a sequence selected from the group consisting of SEQ ID NO 72, SEQ ID NO 73 and SEQ ID NO 74.

6. The probe of claim 2 wherein said nucleotide sequence includes the entire sequence selected from the group consisting of SEQ ID NO 72, SEQ ID NO 73 and SEQ ID NO 74.

7. The probe of claim 1 further comprising five sequential additional nucleotides on either side or both sides of said sequential nucleotides.

8. The probe of claim 2 further comprising five sequential additional nucleotides on either side or both sides of said sequential nucleotides.

9. The probe of claim 3 further comprising five sequential additional nucleotides on either side or both sides of said sequential nucleotides.

10. The probe of claim 1 wherein any of said probes is modified with a label.

11. The probe of claim 10 wherein any of said probe is labeled either terminally or internally with biotin, fluorescent dyes, digoxygenin, radioactivity, or acridinium esters.

12. The probe of claim 10 wherein any of said probe is labeled by an enzymatic or chemical modification.

13. The probe of claim 12 wherein said enzymatic modification is by alkaline phosphatases, kinases, horseradish peroxidase, ligases and jack bean urease or polymerases.

14. The probe of claim 12 wherein said probe is modified by phosphorothioate, peptide bonds, phosphodiester bonds or a combination thereof in the sugar-phosphate backbone of the molecule.

15. The use of the probe of claim 1 in a template dependent assay including hybridization, primer extension, polymerase chain reaction (PCR), nucleic acid sequence-based amplification (NASBA) and strand displacement amplification (SDA), Cycling Probe Reaction (CPR), Ligase Chain Reaction (LCR), or Gapped Ligase Chain Reaction (G-LCR).


SEQUENCE LISTING (74 sequences)

SEQ ID NO: 1 - protein, 553 aa, Methanococcus Vannielii 2187
  CHAIN
  ref <301>: AUTHORS: Cram, D.S., Sherf, B.A., Libby, R.T., Mattalinao, R.I.
  ref <302>: TITLE: Structure and expression of the genes, mcrBDCGA, which encode the subunits of component C of methyl coenzyme M Reductase in Methanococcus vannielii
  ref <303>: JOURNAL: Proc. Natl. Acad. Sci. U.S.A.
  ref <304>: VOLUME: 84
  ref <305>: ISSUE:
  ref <306>: PAGES: 3992 - 3996
  ref <307>: DATE: 1987
  ref <308>: DATABASE ACCESSION NUMBER: AAA72598
  ref <309>: DATABASE ENTRY DATE: 1987-08-25
MEAEKRLFLKALKEKFEEDPKEKYTKFYTYGGWEQSVRKREFVAANEKVLAEKRQGVPLYNPDIGVPLGQRKLMPYKLSGTDSYCEGDDLHFMNNAAIQQLWDDIRRTVVVGMDTAHSVLEKRLGVEVTPETINEYMHTINHALSGGAVVQEHMVEVHPSLAWDSYARIFTGDDELAAELDSRFLIDINKLFPAEQAEALKKAIGKKTYQVSRVPSLVGRVCDGGTISRWSAMQIGMSFITAYKLCAGEAATADFSYASKHADVIQMGNALPGRRARGPNEPGGIQFGILSDVVQTTRVSDDPVEQSLEVVAAGAALYDQIWLGAYMSGGVGFTQYATAAYTDDILDDFSYYALDYVEKKYGRMGTKATMDVVEDIASEVTLYSLEQYDEYPALLEDHFGGSQRAAVAAAASGIGVCMATGNSNAGVNGWYLSQILHKEYHSRLGFYGYDLQDQCGASNSLAIRNDESAPLILRGPNYPNYAMNVGHQGEYAGIAQAAHSARGDAFAMSALIKVAFADPMLVFDFSKPRKEFARGALREFDAAGERDVILPAK

SEQ ID NO: 2 - protein, 7 aa, Methanococcus Vannielii 2187
  CHAIN 329 - 335
VGFTQYA

SEQ ID NO: 3 - protein, 10 aa, Methanococcus Vannielii 2187
  CHAIN 445 - 454
GFYGYDLQDQ

SEQ ID NO: 4 - protein, 7 aa, Methanococcus Vannielii 2187
  CHAIN 475 - 481
GPNYPNY

SEQ ID NO: 5 - protein, 554 aa, Methanothermus fervidus 2180
  CHAIN
  ref <301>: AUTHORS: Lehmacher, A., Klenk, H.P.
  ref <302>: TITLE: Characterization and phylogeny of mcrII, a gene cluster encoding an isoenzyme of methyl coenzyme M reducatase from hyperthermophilic Methanothermus fervidus
  ref <303>: JOURNAL: Mol. Gen. Genet.
  ref <304>: VOLUME: 243
  ref <305>: ISSUE: 2
  ref <306>: PAGES: 198 - 206
  ref <307>: DATE: 1994
  ref <308>: DATABASE ACCESSION NUMBER: CAA50044
  ref <309>: DATABASE ENTRY DATE: 1993-01-21
MNKKNKKLFLEALEKKFKGESPEEKKTTFYCFGGWKQSERKREFVEYAKKLAKKRGIPFYNPDIGVPLGQRKLMAYRISGTDAYVEGDDLHFVNNAAIQQMVDDIKRTVIVGMDTAHAVLEKRLGVEVTPETINEYMETINHALPGGAVVQEHMVEVHPGLVDDCYAKIFTGNDELADELDKRVLIDINKEFPEEQAEMLKKYIGNRTYQVNRVPTIVVRCCDGGTVSRWSAMQIGMSFISAYKLCAGEAAIADFSFAAKHADVIEMGTILPGRRARGPNEPGGIPFGVFADIIQTSRVSDDPARISLEVIGAAATLYDQVWLGSYMSGGVGFTQYASATWTDDILDDFVWWGAEYVEDKYGFCGVKPSMEVVKDIATEVTLYGLEQYEEYPTLLEDHFGGSQRAAVVAAAAGCSTAFATGNSNAGINAWYLSQILHKEGHSRLGFYGYDLQDQCGASNSLSIRSDEGLVHELRGPNYPNYAMNVGHQPEYAGIAQAPHAARGDAFVVNPLIKVAFADNDLSFDFRWPRKEIARGALREFMPDGERTLIIPASK

SEQ ID NO: 6 - protein, 7 aa, Methanothermus fervidus 2180
  CHAIN 331 - 337
VGFTQYA

SEQ ID NO: 7 - protein, 10 aa, Methanothermus fervidus 2180
  CHAIN 445 - 454
GFYGYDLQDQ

SEQ ID NO: 8 - protein, 7 aa, Methanothermus fervidus 2180
  CHAIN 475 - 481
GPNYPNY

SEQ ID NO: 9 - protein, 553 aa, Methanopyrus kandleri 2320
  CHAIN
  ref <301>: AUTHORS: Nolling, I., Elfner, A.,Palmer, J.R., Steigerwald, V.J., Pihl, T.D., Lake, J.A., Reeve, J.N.
  ref <302>: TITLE: Phylogeny of Methanopyrus kandleri based on methyl coenzyme M reductase operons
  ref <303>: JOURNAL: Int. J. Syst. Bacteriol.
  ref <304>: VOLUME: 46
  ref <305>: ISSUE: 4
  ref <306>: PAGES: 1170 - 1173
  ref <307>: DATE: 1996
  ref <308>: DATABASE ACCESSION NUMBER: U57340
  ref <309>: DATABASE ENTRY DATE: 1996-05-01
MSSAEEKLFMKALKEKFEESPEEKYTKFYIFGGWKQSERKKEFKEWADKIVEERGVPHYNPDIGVPLGQRKLMSYQVSGTDVFVEGDDLTFVNNAAMQQMWDDIRRTVIVGMDTAHRVLERRLGKEVTPETINEYMETLNHALPGGAVVQEHMVEIHPGLTWDCYAKIITGDLELADEIDDKFLIDIEKLFPEEQAEQLIKAIGNRTYQVCRMPTIVGHVCDGATMYRWAAMQIAMSFICAYKIAAGEAAVSDFAFASKHAEVINMGEMLPARRARGENEPGGVPFGVLADCVQTMRKYPDDPAKVALEVIAAGAMLYDQIWLGSYMSGGVGFTQYATAVYPDNILDDYVYYGLEYVEDKYGIAEAEPSMDVVKDVATEVTLYGLEQYERYPAAMETHFGGSQRAAVCAAAAGCSTAFATGHAQAGLNGWYLSQILHKEGQGRLGFYGYALQDQCGAANSLSVRSDEGLPLELRGPNYPNYAMNVGHLGEYAGIVQAAHAARGDAFCVHPVIKVAFADENLVFDFTEPRKEFAKGALREFEPAGERDLIVPAE

SEQ ID NO: 10 - protein, 7 aa, Methanopyrus kandleri 2320
  CHAIN 331 - 337
VGFTQYA

SEQ ID NO: 11 - protein, 10 aa, Methanopyrus kandleri 2320
  CHAIN 445 - 454
GFYGYALQDQ

SEQ ID NO: 12 - protein, 7 aa, Methanopyrus kandleri 2320
  CHAIN 475 - 481
GPNYPNY

SEQ ID NO: 13 - protein, 550 aa, Methanobacterium thermoautotrophicum 2166
  CHAIN
  ref <301>: AUTHORS: Knaub, S., Klein, A.
  ref <302>: TITLE: Specific transcription of cloned Methanobacterium thermoautotrophicum transcription units by homologous RNA polymerase in vitro
  ref <303>: JOURNAL: Nucleic Acids Res.
  ref <304>: VOLUME: 18
  ref <305>: ISSUE: 6
  ref <306>: PAGES: 1441 - 1446
  ref <307>: DATE: 1990
  ref <308>: DATABASE ACCESSION NUMBER: X07794
  ref <309>: DATABASE ENTRY DATE: 1988-05-30
MADKLFINALKKKFEESPEEKKTTFYTLGGWKQSERKTEFVNAGKEVAAKRGIPQYNPDIGTPLGQRVLMPYQVSTTDTYVEGDDLHFVNNAAMQQMWDDIRRTVIVGLNHAHAVIEKRLGKEVTPETITHYLETVNHAMPGAAVVQEHMVETHPALVADSYVKVFTGNDEIADEIDPAFVIDINKQFPEDQAETLKAEVGDGIWQVVRIPTIVSRTCDGATTSRWSAMQIGMSMISAYKQAAGEAATGDFAYAAKHAEVIHMGTYLPVRRARGENEPGGVPFGYLADICQSSRVNYEDPVRVSLDVVATGAMLYDQIWLGSYMSGGVGFTQYATAAYTDNILDDFTYFGKEYVEDKYGLCEAPNNMDTVLDVATEVTFYGLEQYEEYPALLEDQFGGSQRAAVVAAAAGCSTAFATGNAQTGLSGWYLSMYLHKEQHSRLGFYGYDLQDQCGASNVFSIRGDEGLPLELRGPNYPNYAMNVGHQGEYAGISQAPHAARGDAFVFNPLVKIAFADDNLVFDFTNVRGEFAKGALREFEPAGERALITPAK

SEQ ID NO: 14 - protein, 7 aa, Methanobacterium thermoautotrophicum 2166
  CHAIN 328 - 334
VGFTQYA

SEQ ID NO: 15 - protein, 10 aa, Methanobacterium thermoautotrophicum 2166
  CHAIN 442 - 451
GFYGYDLQDQ

SEQ ID NO: 16 - protein, 7 aa, Methanobacterium thermoautotrophicum 2166
  CHAIN 472 - 478
GPNYPNY

SEQ ID NO: 17 - protein, 570 aa, Methanosarcina barkeri 2208
  CHAIN
  ref <301>: AUTHORS: Bokranz, M., Klein, A.
  ref <302>: TITLE: Nucleotide sequence of the methyl coenzyme M reductase gene cluster from Methanosarcina barkeri
  ref <303>: JOURNAL: Nucleic Acids Res.
  ref <304>: VOLUME: 15
  ref <305>: ISSUE: 10
  ref <306>: PAGES: 4350 - 4351
  ref <307>: DATE: 1987
  ref <308>: DATABASE ACCESSION NUMBER: E29525
  ref <309>: DATABASE ENTRY DATE: 1996-10-04
MAADIFSKFKKDMEVKFAQEFGSNKQTGGDITDKTAKFLRLGPEQDPRKVEMIKAGKEIAEKRGIAFYNPMMHSGAPLGQRAITPYTISGTDIVCEPDDLHYVNNAAMQQMWDDIRRTCIVGLDMAHETLEKRLGKEVTPETINHYLEVLNHAMPGAAVVQEMMVETHPALVDDCYVKVFTGDDALADEIDKQFLIDINKEFSEEQAAQIKASIGKTSWQAIHIPTIVSRTTDGAQTSRWAAMQIGMSFISAYAMCAGEAAVADLSFAAKHAALVSMGEMLPARRARGPNEPGGLSFGHLSDIVQTSRVSEDPAKIALEVVGAGCMLYDQIWLGSYMSGGVGFTQYATAAYTDDILDNNTYYDVDYINDKYNGAATVGKDNKVKASLEVVKDIATESTLYGIETYEKFPTALEDHFGGSQRATVLAAAAGVACSLATGNANAGLSGWYLSMYLHKEAWGRLGFFGFDLQDQCGATNVLSYQGDEGLPDELRGPNYPNYAMNVGHQGGYAGIAQAAHSGRGDAFTVNPLLKVCFADDLLPFNFAEPRREFGRGAIREFVPAGERSLVIPAK

SEQ ID NO: 18 - protein, 7 aa, Methanosarcina barkeri 2208
  CHAIN 341 - 347
VGFTQYA

SEQ ID NO: 19 - protein, 10 aa, Methanosarcina barkeri 2208
  CHAIN 462 - 471
GFFGFDLQDQ

SEQ ID NO: 20 - protein, 7 aa, Methanosarcina barkeri 2208
  CHAIN 492 - 498
GPNYPNY

SEQ ID NO: 21 - protein, 7 aa, unknown
  CHAIN 78 - 84: obtained from marine sediment near the Cascadia Margin
  ref <301>: AUTHORS: Bidle,K.A., Kastner,M. and Bartlett,D.H.
  ref <302>: TITLE: A phylogenetic analysis of microbial communities associated with methane hydrate containing marine fluids and sediments in the Cascadia margin (ODP site 892B)
  ref <303>: JOURNAL: FEMS Microbiol. Lett.
  ref <304>: VOLUME: 1
  ref <305>: ISSUE: 177
  ref <306>: PAGES: 101 - 108
  ref <307>: DATE: 1999
  ref <308>: DATABASE ACCESSION NUMBER: AAD45633
  ref <309>: DATABASE ENTRY DATE: 1999-01-19
VGFTQYS

SEQ ID NO: 22 - protein, 7 aa, unknown
  CHAIN 8 - 14: environmental clone amplified from rice field soil
  ref <301>: AUTHORS: Lueders,T., Chin,K.J., Conrad,R. and Friedrich,M.
  ref <302>: TITLE: Molecular analyses of methyl-coenzyme M reductase alpha-subunit mcrA) genes in rice field soil and enrichment cultures reveal the methanogenic phenotype of a novel archaeal lineage
  ref <303>: JOURNAL: Environ. Microbiol.
  ref <304>: VOLUME: 3
  ref <305>: ISSUE: 3
  ref <306>: PAGES: 194-204
  ref <307>: DATE: 2001
  ref <308>: DATABASE ACCESSION NUMBER: AAK16842
  ref <309>: DATABASE ENTRY DATE: 2000-10-17
VGFTMYA

SEQ ID NO: 23 - protein, 5 aa, unknown
  CHAIN 153 - 157: environmental clone amplified from rice field soil
  ref <301>: AUTHORS: Lueders,T., Chin,K.J., Conrad,R. and Friedrich,M.
  ref <302>: TITLE: Molecular analyses of methyl-coenzyme M reductase alpha-subunit mcrA) genes in rice field soil and enrichment cultures reveal the methanogenic phenotype of a novel archaeal lineage
  ref <303>: JOURNAL: Environ. Microbiol.
  ref <304>: VOLUME: 3
  ref <305>: ISSUE: 3
  ref <306>: PAGES: 194-204
  ref <307>: DATE: 2001
  ref <308>: DATABASE ACCESSION NUMBER: AAK16853
  ref <309>: DATABASE ENTRY DATE: 2000-10-17
GANYP

SEQ ID NO: 24 - protein, 10 aa, unknown
  CHAIN 123 - 132: environmental clone amplified from rice field soil
  ref <301>: AUTHORS: Lueders,T., Chin,K.J., Conrad,R. and Friedrich,M.
  ref <302>: TITLE: Molecular analyses of methyl-coenzyme M reductase alpha-subunit mcrA) genes in rice field soil and enrichment cultures reveal the methanogenic phenotype of a novel archaeal lineage
  ref <303>: JOURNAL: Environ. Microbiol.
  ref <304>: VOLUME: 3
  ref <305>: ISSUE: 3
  ref <306>: PAGES: 194-204
  ref <307>: DATE: 2001
  ref <308>: DATABASE ACCESSION NUMBER: AAK16853
  ref <309>: DATABASE ENTRY DATE: 2000-10-17
GFYGYDLQDQ

SEQ ID NO: 25 - protein, 10 aa, unknown
  CHAIN 123 - 132: environmental clone amplified from rice field soil
  ref <301>: AUTHORS: Lueders,T., Chin,K.J., Conrad,R. and Friedrich,M.
  ref <302>: TITLE: Molecular analyses of methyl-coenzyme M reductase alpha-subunit mcrA) genes in rice field soil and enrichment cultures reveal the methanogenic phenotype of a novel archaeal lineage
  ref <303>: JOURNAL: Environ. Microbiol.
  ref <304>: VOLUME: 3
  ref <305>: ISSUE: 3
  ref <306>: PAGES: 194-204
  ref <307>: DATE: 2001
  ref <308>: DATABASE ACCESSION NUMBER: AAK16858
  ref <309>: DATABASE ENTRY DATE: 2000-10-17
GFFGYDLQGQ

SEQ ID NO: 26 - protein, 7 aa, unknown
  CHAIN 8 - 14: environmental clone amplified from rice field soil
  ref <301>: AUTHORS: Lueders,T., Chin,K.J., Conrad,R. and Friedrich,M.
  ref <302>: TITLE: Molecular analyses of methyl-coenzyme M reductase alpha-subunit mcrA) genes in rice field soil and enrichment cultures reveal the methanogenic phenotype of a novel archaeal lineage
  ref <303>: JOURNAL: Environ. Microbiol.
  ref <304>: VOLUME: 3
  ref <305>: ISSUE: 3
  ref <306>: PAGES: 194-204
  ref <307>: DATE: 2001
  ref <308>: DATABASE ACCESSION NUMBER: AAK16861
  ref <309>: DATABASE ENTRY DATE: 2000-10-17
VGFAQYA

SEQ ID NO: 27 - protein, 7 aa, unknown
  CHAIN 8 - 14: environmental clone amplified from rice field soil
  ref <301>: AUTHORS: Lueders,T., Chin,K.J., Conrad,R. and Friedrich,M.
  ref <302>: TITLE: Molecular analyses of methyl-coenzyme M reductase alpha-subunit mcrA) genes in rice field soil and enrichment cultures reveal the methanogenic phenotype of a novel archaeal lineage
  ref <303>: JOURNAL: Environ. Microbiol.
  ref <304>: VOLUME: 3
  ref <305>: ISSUE: 3
  ref <306>: PAGES: 194-204
  ref <307>: DATE: 2001
  ref <308>: DATABASE ACCESSION NUMBER: AAK16862
  ref <309>: DATABASE ENTRY DATE: 2000-10-17
VGFAQYA

SEQ ID NO: 28 - protein, 10 aa, unknown
  CHAIN 129 - 138: environmental clone amplified from rice field soil
  ref <301>: AUTHORS: Lueders,T., Chin,K.J., Conrad,R. and Friedrich,M.
  ref <302>: TITLE: Molecular analyses of methyl-coenzyme M reductase alpha- subunit mcrA) genes in rice field soil and enrichment cultures reveal the methanogenic phenotype of a novel archaeal lineage
  ref <303>: JOURNAL: Environ. Microbiol.
  ref <304>: VOLUME: 3
  ref <305>: ISSUE: 3
  ref <306>: PAGES: 194-204
  ref <307>: DATE: 2001
  ref <308>: DATABASE ACCESSION NUMBER: AAK16853
  ref <309>: DATABASE ENTRY DATE: 2000-10-17
GFFGYDLQDQ

SEQ ID NO: 29 - protein, 10 aa, Methanopyrus kandleri
  CHAIN 445 - 454
  ref <301>: AUTHORS: Lake,J.A. and Reeve,J.N.
  ref <302>: TITLE: Phylogeny of Methanopyrus kandleri based on methyl coenzyme M reductase operons
  ref <303>: JOURNAL: Int. J. Syst. Bacteriol.
  ref <304>: VOLUME: 46
  ref <305>: ISSUE: 4
  ref <306>: PAGES: 1170 - 1173
  ref <307>: DATE: 1996
  ref <308>: DATABASE ACCESSION NUMBER: Q49605
  ref <309>: DATABASE ENTRY DATE: 1999-07-15
GFYGYALQDQ

SEQ ID NO: 30 - protein, 7 aa, Artificial Sequence
VGFTQYA

SEQ ID NO: 31 - protein, 7 aa, Artificial Sequence
VGFTQYS

SEQ ID NO: 32 - protein, 7 aa, Artificial Sequence
VGFTMYS

SEQ ID NO: 33 - protein, 7 aa, Artificial Sequence
VGFTMYA

SEQ ID NO: 34 - protein, 7 aa, Artificial Sequence
VGFAQYA

SEQ ID NO: 35 - protein, 7 aa, Artificial Sequence
VGFAQYS

SEQ ID NO: 36 - protein, 7 aa, Artificial Sequence
VGFAMYS

SEQ ID NO: 37 - protein, 7 aa, Artificial Sequence
VGFAMYA

SEQ ID NO: 38 - protein, 7 aa, Artificial Sequence
VGFVQYA

SEQ ID NO: 39 - protein, 7 aa, Artificial Sequence
VGFVQYS

SEQ ID NO: 40 - protein, 7 aa, Artificial Sequence
VGFVMYS

SEQ ID NO: 41 - protein, 7 aa, Artificial Sequence
VGFVMYA

SEQ ID NO: 42 - protein, 7 aa, Artificial Sequence
VGFGQYA

SEQ ID NO: 43 - protein, 7 aa, Artificial Sequence
VGFGQYS

SEQ ID NO: 44 - protein, 7 aa, Artificial Sequence
VGFGMYS

SEQ ID NO: 45 - protein, 7 aa, Artificial Sequence
VGFGMYA

SEQ ID NO: 46 - protein, 7 aa, Artificial Sequence
GPNYPNY

SEQ ID NO: 47 - protein, 7 aa, Artificial Sequence
GANYPNY

SEQ ID NO: 48 - protein, 10 aa, Artificial Sequence
GFFGFDLQDQ

SEQ ID NO: 49 - protein, 10 aa, Artificial Sequence
GFFGFDLQGQ

SEQ ID NO: 50 - protein, 10 aa, Artificial Sequence
GFFGFALQDQ

SEQ ID NO: 51 - protein, 10 aa, Artificial Sequence
GFFGFALQGQ

SEQ ID NO: 52 - protein, 10 aa, Artificial Sequence
GFFGYDLQDQ

SEQ ID NO: 53 - protein, 10 aa, Artificial Sequence
GFFGYDLQGQ

SEQ ID NO: 54 - protein, 10 aa, Artificial Sequence
GFFGYALQDQ

SEQ ID NO: 55 - protein, 10 aa, Artificial Sequence
GFFGYALQGQ

SEQ ID NO: 56 - protein, 10 aa, Artificial Sequence
GFFGHDLQDQ

SEQ ID NO: 57 - protein, 10 aa, Artificial Sequence
GFFGHDLQGQ

SEQ ID NO: 58 - protein, 10 aa, Artificial Sequence
GFFGHALQDQ

SEQ ID NO: 59 - protein, 10 aa, Artificial Sequence
GFFGHALQGQ

SEQ ID NO: 60 - protein, 10 aa, Artificial Sequence
GFYGFDLQDQ

SEQ ID NO: 61 - protein, 10 aa, Artificial Sequence
GFYGFDLQGQ

SEQ ID NO: 62 - protein, 10 aa, Artificial Sequence
GFYGFALQDQ

SEQ ID NO: 63 - protein, 10 aa, Artificial Sequence
GFYGFALQGQ

SEQ ID NO: 64 - protein, 10 aa, Artificial Sequence
GFYGYDLQDQ

SEQ ID NO: 65 - protein, 10 aa, Artificial Sequence
GFYGYDLQGQ

SEQ ID NO: 66 - protein, 10 aa, Artificial Sequence
GFYGYALQDQ

SEQ ID NO: 67 - protein, 10 aa, Artificial Sequence
GFYGYALQGQ

SEQ ID NO: 68 - protein, 10 aa, Artificial Sequence
GFYGHDLQDQ

SEQ ID NO: 69 - protein, 10 aa, Artificial Sequence
GFYGHDLQGQ

SEQ ID NO: 70 - protein, 10 aa, Artificial Sequence
GFYGHALQDQ

SEQ ID NO: 71 - protein, 10 aa, Artificial Sequence
GFYGHALQGQ

SEQ ID NO: 72 - DNA, 20 bp, Artificial Sequence
gttggtttyrgtmwrtayds

SEQ ID NO: 73 - DNA, 20 bp, Artificial Sequence
tartttggrtartttgstcc

SEQ ID NO: 74 - DNA, 29 bp, Artificial Sequence
tgrycytgtarrkcrwrtccrwaraatcc